(19) United States
(12) Patent Application Publication
(10) Pub. No.: US 2026/0151340 A1
(43) Pub. Date: Jun. 4, 2026

(54) PEPTIDE-LIPID CONJUGATES AND APPLICATION THEREOF

(71) Applicant: Vacino Biotech Co., Ltd., Taipei (TW)

(72) Inventors: Jia-Ming CHANG, Taipei (TW); I-Hua LIU, Taipei (TW)

(21) Appl. No.: 19/408,584

(22) Filed: Dec. 4, 2025

Related U.S. Application Data
(60) Provisional application No. 63/727,681, filed on Dec. 4, 2024.

(51) Int. Cl. A61K 9/1275; A61K 31/7105; C07K 1/107

(52) U.S. Cl. CPC A61K 9/1275; A61K 31/7105; C07K 1/1077

(57) ABSTRACT

A peptide-lipid conjugate is provided. The conjugate includes a peptide selected from SEQ ID NO: 1 to SEQ ID NO: 29 and a hydrophobic carbon chain conjugated to the peptide. The conjugate further includes a lipid-based carrier encapsulating at least one reagent, and the hydrophobic carbon chain anchors into a lipid layer of the lipid-based carrier. The peptide can bind to a transferrin receptor (TfR), and binding of the peptide to transferrin receptors on cells of a tissue barrier induces transcytosis of the cells, thereby transporting the conjugate across the tissue barrier. Thus, the conjugate can serve as a drug delivery system.

### CROSS-REFERENCE TO RELATED APPLICATIONS

This application claims priority to and the benefit of U.S. Provisional Application No. 63/727,681, filed on Dec. 4, 2024, the disclosure of which is incorporated by reference in its entirety.

### SUBMISSION OF SEQUENCE LISTING AS ASCII TEXT FILE

This application includes an electronically submitted sequence listing in XML format. The XML file contains a sequence listing entitled “P25-0296US_Sequence_Listing.xml” which was created on Dec. 2, 2025 and is 25,590 bytes in size. The sequence listing contained in this XML file is part of the specification and is hereby incorporated by reference herein in its entirety.

### BACKGROUND OF THE INVENTION

### 1. Field of the Invention

The present invention relates to peptide-lipid conjugates capable of crossing tissue barriers and methods of using these peptide-lipid conjugates for delivering reagents across tissue barriers.

### 2. Description of the Prior Art

Tissue barriers in animals and humans are generally understood to function to restrict or regulate the passage of substances. Such barriers are present in various tissues and organs to maintain the environmental stability (homeostasis) of specific regions of the body and/or to protect vital organs from the influence of external substances. Examples of tissue barriers include, without limitation, blood-brain barrier (BBB), gastrointestinal mucosal barrier, a blood retina barrier (BRB), a blood-thymus barrier (BTB), a blood-testis barrier (BTB), and a blood-placenta barrier (BPB).

The BBB is located in the central nervous system and is primarily formed by brain microvascular endothelial cells (BMECs). The BBB serves to protect the brain from exogenous substances and to regulate the transport of specific molecules, such as oxygen and nutrients, into the brain in order to maintain normal brain function. However, it has been reported that the presence of the BBB significantly limits the entry of therapeutic agents into the brain. Clinical studies have indicated that less than about 0.1% of systemically administered neurological drugs are able to cross the BBB and reach the brain. Accordingly, the BBB presents a substantial barrier to drug delivery, and this limitation has long been recognized as a major challenge in the treatment of central nervous system (CNS) diseases.

Lipid nanoparticles (LNPs) have been reported to be predominantly distributed to the liver and, to a lesser extent, to the spleen following systemic administration. In particular, it is known that LNPs are captured by the liver via the low-density lipoprotein (LDL) receptor, because serum apolipoprotein E (ApoE), a ligand of the LDL receptor, binds to cholesterol present on the surface of the LNPs upon entry into the circulation. It has further been described that parameters such as lipid composition, particle size, and pKa can affect the organ biodistribution of LNPs. For example, LNPs rich in cholesterol have been shown to predominantly accumulate in the liver, LNPs having a net positive charge tend to accumulate in the lung, and LNPs containing an anionic helper lipid and exhibiting a net negative charge tend to accumulate in the spleen. In addition, LNPs having a particle size of less than about 100 nm have been reported to readily and effectively penetrate capillary beds and to accumulate in the liver and spleen (Vasileva et al., 2024). Notwithstanding such developments, it remains challenging for LNPs to penetrate tissue barriers, such as BBB. Approaches that have been proposed to address this limitation include, for example, direct intracerebral injection (Vasileva et al. (2024) Composition of lipid nanoparticles for targeted delivery: application to mRNA therapeutics. Front. Pharmacol. 15:1466337) and transient opening of the BBB using ultrasound to facilitate drug delivery. Furthermore, the circulation half-life of LNPs has been reported to be on the order of about half an hour, due at least in part to rapid clearance by liver Kupffer cells, which remove circulating particles and thereby limit the therapeutic effectiveness of LNP-based formulations (Zhang et al (2025) Investigating the stability of RNA-lipid nanoparticles in biological fluids: Unveiling its crucial role for understanding LNP performance. J. Control Release 381: 113559).

Thus, there is a persistent need for improved LNP formulations or delivery strategies that overcome these deficiencies, particularly fast delivery to the target within the circulation half-life and enabling effective non-invasive delivery across formidable tissue barriers.

### SUMMARY OF THE INVENTION

The present invention is based, at least in part, on the inventors' previous study that peptides comprising the amino acid sequence of SEQ ID NO: 1 to SEQ ID NO: 29 have binding affinity to transferrin receptors (TfRs). These peptides can effectively bind to TfRs on the cells of tissue barriers and induce transcytosis of the cells to allow the peptides and reagents conjugated to the peptides together to cross the tissue barriers. In order to improve the efficiency of drug delivery, these peptides are modified by hydrophobic carbon chains to allow these peptides to combine with a lipid-based carrier more effectively. Therefore, the present invention provides a peptide-lipid conjugate comprising a peptide consisting of an amino acid sequence selected from the group consisting of SEQ ID NO: 1 to SEQ ID NO: 29 and a hydrophobic carbon chain conjugated to the peptide.

In some embodiments, the peptide-lipid conjugate of the present invention further comprises a lipid-based carrier, and the hydrophobic carbon chain anchors into a lipid layer of the lipid-based carrier to form a peptide-lipid-(lipid-based carrier) conjugate. In some embodiments, at least one reagent is encapsulated in the lipid-based carrier. These peptides can transport the at least one reagent encapsulated in the lipid-based carrier to a target by binding to TfRs on cells of the target. In some embodiments, binding of the peptides to TfRs on cells of a tissue barrier induces transcytosis of the cell, thereby transport the peptide-lipid conjugate and/or the peptide-lipid-(lipid-based carrier) conjugate across the tissue barrier. Therefore, the peptide-lipid conjugate and the peptide-lipid-(lipid-based carrier) conjugate disclosed herein can serve as a drug delivery system, especially a drug delivery system across a tissue barrier, such as BBB.

Those skilled in the art will recognize, or be able to ascertain using no more than routine experimentation, many equivalents to the specific embodiments of the invention described herein. Such equivalents are intended to be encompassed by the following embodiments.

Embodiment 1. A peptide-lipid conjugate, comprising:
a peptide consisting of an amino acid sequence selected from the group consisting of SEQ ID NO: 1 to SEQ ID NO: 29; and
a hydrophobic carbon chain conjugated to the peptide.

Embodiment 2. The peptide-lipid conjugate of Embodiment 1, wherein a first carbon or a last carbon of the hydrophobic carbon chain is conjugated to a lysine of the peptide.

Embodiment 3. The peptide-lipid conjugate of Embodiment 1 or 2, wherein the first carbon or the last carbon of the hydrophobic carbon chain is conjugated to a lysine of the peptide via a triazole moiety.

Embodiment 4. The peptide-lipid conjugate of any one of Embodiments 1 to 3, wherein the hydrophobic carbon chain comprises at least 6 carbons.

Embodiment 5. The peptide-lipid conjugate of any one of Embodiments 1 to 4, wherein the hydrophobic carbon chain is further conjugated to polyethylene glycol.

Embodiment 6. The peptide-lipid conjugate of any one of Embodiments 1 to 5, wherein the hydrophobic carbon chain is selected from dodecyne, cholesterol, and cholesterol-PEG.

Embodiment 7. The peptide-lipid conjugate of any one of Embodiments 1 to 6, further comprising a lipid-based carrier, wherein the hydrophobic carbon chain anchors into a lipid layer of the lipid-based carrier.

Embodiment 8. The peptide-lipid conjugate of Embodiment 7, wherein the lipid-based carrier is selected form liposome, PEGylated liposome, cationic liposome, immunoliposome, transfersome, ethosome, niosome, virosome, nanoparticle, lipid nanoparticle, lipid nanoemulsion, microemulsion, pickering emulsion, micelle, cubosome, hexosome, spongosome, lipid-polymer hybrid nanoparticle, exosome, endosome, and extracellular vesicle.

Embodiment 9. The peptide-lipid conjugate of Embodiment 7 or 8, wherein at least one reagent is encapsulated in the lipid-based carrier.

Embodiment 10. The peptide-lipid conjugate of Embodiment 9, wherein the at least one reagent is selected from siRNA, shRNA, microRNA, double-stranded RNA, single-stranded RNA, DNA, aptamers, peptides, proteins, small chemical molecules, large chemical molecules, viral particles or fragments thereof, dendrimers, ligands, eukaryotic cells or fragments thereof, and prokaryotic cells or fragments thereof.

Embodiment 11. A method of transporting at least one reagent to a target, comprising binding a peptide-lipid-(lipid-based carrier) conjugate to a transferrin receptor, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating the at least one reagent;
   wherein a first carbon or a last carbon of the hydrophobic carbon chain is conjugated to a lysine of the peptide, and the hydrophobic carbon chain anchors into a lipid layer of the lipid-based carrier.

Embodiment 12. The method of Embodiment 11, wherein the hydrophobic carbon chain comprises at least 6 carbons.

Embodiment 13. The method of Embodiment 11 or 12, wherein the hydrophobic carbon chain is further conjugated to polyethylene glycol.

Embodiment 14. The method of any one of Embodiments 11 to 13, wherein the lipid-based carrier is selected form liposome, PEGylated liposome, cationic liposome, immunoliposome, transfersome, ethosome, niosome, virosome, nanoparticle, lipid nanoparticle, lipid nanoemulsion, microemulsion, pickering emulsion, micelle, cubosome, hexosome, spongosome, lipid-polymer hybrid nanoparticle, exosome, endosome, and extracellular vesicle.

Embodiment 15. The method of any one of Embodiments 11 to 14, wherein the at least one reagent is selected from siRNA, shRNA, microRNA, double-stranded RNA, single-stranded RNA, DNA, aptamers, peptides, proteins, small chemical molecules, large chemical molecules, viral particles or fragments thereof, dendrimers, ligands, eukaryotic cells or fragments thereof, and prokaryotic cells or fragments thereof.

Embodiment 16. The method of any one of Embodiments 11 to 15, wherein the at least one reagent has to be transported across a tissue barrier to arrive at the target, and the transferrin receptor is located on a cell of the tissue barrier.

Embodiment 17. The method of any one of Embodiments 11 to 16, wherein binding of the peptide to the transferrin receptor induces transcytosis of the cells, thereby transporting the peptide-lipid-(lipid-based carrier) conjugate across the tissue barrier to arrive at the target.

Embodiment 18. The method of Embodiment 16 or 17, wherein the tissue barrier is one of a blood-brain barrier (BBB), a mucosal barrier, a gastrointestinal barrier, a blood retina barrier (BRB), a blood-thymus barrier (BTB), a blood-testis barrier (BTB), and a blood-placenta barrier (BPB).

Embodiment 19. The method of any one of Embodiments 11 to 18, wherein the target is a brain cell.

Embodiment 20. The method of any one of Embodiments 11 to 19, wherein the tissue barrier is blood-brain barrier, and the target is a brain cell.

Embodiment 21. The method of any one of Embodiments 11 to 15, wherein the target is a cell expressing the transferrin receptor, the transferrin receptor is located on a cell membrane of the cell, and binding of the peptide-lipid-(lipid-based carrier) conjugate to the transferrin receptor induces transcytosis of the cells, thereby transporting the peptide-lipid-(lipid-based carrier) conjugate across the cell membrane to arrive into the cell.

Embodiment 22. The method of any one of Embodiment 11 to 15 or 21, wherein the target is a cancer cell.

Embodiment 23. The method of Embodiment 22, wherein the cancer is hepatocellular carcinoma, breast cancer, lung cancer, colon cancer, brain cancer, glioma, prostate cancer, ovarian cancer, or leukemia.

Embodiment 24. The method of any one of Embodiment 11 to 15 or 21, wherein the target is a tissue cell.

Embodiment 25. The method of Embodiment 21, wherein the tissue is skin, tonsil, tongue, oesophagus, cervix, kidney, placenta, pancreas, testis, anterior pituitary, stomach, breast, or liver.

Embodiment 26. The method of any one of Embodiments 11 to 25, wherein the target is in vivo or in vitro.

Embodiment 27. A method of transporting at least one reagent across a tissue barrier of a subject, comprising administering to the subject a peptide-lipid-(lipid-based carrier) conjugate, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating the at least one reagent;
   wherein a first carbon or a last carbon of the hydrophobic carbon chain is conjugated to a lysine of the peptide, and the hydrophobic carbon chain anchors into a lipid layer of the lipid-based carrier.

Embodiment 28. The method of Embodiment 27, wherein the tissue barrier is one of a blood-brain barrier (BBB), a mucosal barrier, a gastrointestinal barrier, a blood retina barrier (BRB), a blood-thymus barrier (BTB), a blood-testis barrier (BTB), and a blood-placenta barrier (BPB).

Embodiment 29. The method of Embodiment 27 or 28, wherein cells of the tissue barrier express transferrin receptors.

Embodiment 30. The method of Embodiment 29, wherein the peptide binds to the transferrin receptor, and the binding of the peptide to the transferrin receptor induces transcytosis of the cells, thereby transporting the peptide-lipid-(lipid-based carrier) conjugate across the tissue barrier.

Embodiment 31. The method of any one of Embodiments 27 to 30, wherein the hydrophobic carbon chain comprises at least 6 carbons.

Embodiment 32. The method of Embodiments 31, wherein the hydrophobic carbon chain is further conjugated to polyethylene glycol.

Embodiment 33. The method of any one of Embodiments 27 to 32, wherein the lipid-based carrier is selected form liposome, PEGylated liposome, cationic liposome, immunoliposome, transfersome, ethosome, niosome, virosome, nanoparticle, lipid nanoparticle, lipid nanoemulsion, microemulsion, pickering emulsion, micelle, cubosome, hexosome, spongosome, lipid-polymer hybrid nanoparticle, exosome, endosome, and extracellular vesicle.

Embodiment 34. The method of any one of Embodiments 27 to 33, wherein the at least one reagent is selected from siRNA, shRNA, microRNA, double-stranded RNA, single-stranded RNA, DNA, aptamers, peptides, proteins, small chemical molecules, large chemical molecules, viral particles or fragments thereof, dendrimers, ligands, eukaryotic cells or fragments thereof, and prokaryotic cells or fragments thereof.

Embodiment 35. The method of any one of Embodiments 27 to 34, wherein the peptide-lipid-(lipid-based carrier) conjugate is administered to the subject by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery.

Embodiment 36. The method of any one of Embodiments 27 to 35, wherein the subject is a mammal.

Embodiment 37. A method of treating or preventing a central nervous system (CNS) disease, comprising administering a subject in need thereof a pharmaceutically effective amount of a peptide-lipid-(lipid-based carrier) conjugate, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating a therapeutic agent of the CNS disease;
   wherein a first carbon or a last carbon of the hydrophobic carbon chain is conjugated to a lysine of the peptide, and the hydrophobic carbon chain anchors into a lipid layer of the lipid-based carrier.

Embodiment 38. The method of Embodiment 37, wherein the hydrophobic carbon chain comprises at least 6 carbons.

Embodiment 39. The method of Embodiment 37 or 38, wherein the hydrophobic carbon chain is further conjugated to polyethylene glycol.

Embodiment 40. The method of any one of Embodiments 37 to 39, wherein the lipid-based carrier is selected form liposome, PEGylated liposome, cationic liposome, immunoliposome, transfersome, ethosome, niosome, virosome, nanoparticle, lipid nanoparticle, lipid nanoemulsion, microemulsion, pickering emulsion, micelle, cubosome, hexosome, spongosome, lipid-polymer hybrid nanoparticle, exosome, endosome, and extracellular vesicle.

Embodiment 41. The method of any one of Embodiments 37 to 40, wherein the peptide of the peptide-lipid-(lipid-based carrier) conjugate binds to a transferrin receptor on a cell of a tissue barrier of the subject, and the binding of the peptide to the transferrin receptor induces transcytosis of the cell, thereby transporting the peptide-lipid-(lipid-based carrier) conjugate across the tissue barrier.

Embodiment 42. The method of Embodiments 41, wherein the tissue barrier is one of a blood-brain barrier, a mucosal barrier, a gastrointestinal barrier, a blood retina barrier (BRB), a blood-thymus barrier (BTB), a blood-testis barrier (BTB), and a blood-placenta barrier (BPB).

Embodiment 43. The method of any one of Embodiments 37 to 42, wherein the subject is a mammal.

Embodiment 44. The method of any one of Embodiments 37 to 43, wherein the peptide-lipid-(lipid-based carrier) conjugate is administered to the subject in need thereof by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery.

Embodiment 45. The method of any one of Embodiments 37 to 44, wherein the CNS disease is one of Alzheimer's disease (AD), Parkinson's disease (PD), cerebrovascular accidents (CVA), ascular-related dementia, Creutzfeldt-Jakob disease (CJD), bovine spongiform encephalopathy (BSE), Traumatic Brain Injury (TBI), multiple sclerosis (MS), amyotrophic lateral sclerosis (ALS), Huntington's chorea, and spinal muscular atrophy (SMA).

Embodiment 46. A peptide-lipid-(lipid-based carrier) conjugate for use in delivering at least one reagent to a target, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating the at least one reagent;
   wherein a first carbon or a last carbon of the hydrophobic carbon chain is conjugated to a lysine of the peptide, and the hydrophobic carbon chain anchors into a lipid layer of the lipid-based carrier.

Embodiment 47. Use of a peptide-lipid-(lipid-based carrier) conjugate for the manufacture of a medicament for delivering at least one reagent to a target, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating the at least one reagent;
   wherein a first carbon or a last carbon of the hydrophobic carbon chain is conjugated to a lysine of the peptide, and the hydrophobic carbon chain anchors into a lipid layer of the lipid-based carrier.

Embodiment 48. The use of Embodiment 46 or 47, wherein the peptide-lipid-(lipid-based carrier) conjugate has to cross a tissue barrier to arrive at the target, and the peptide binds to a transferrin receptor on a cell of the tissue barrier.

Embodiment 49. The use of Embodiment 48, wherein binding of the peptide to the transferrin receptor induces transcytosis of the cells, thereby transporting the peptide-lipid-(lipid-based carrier) conjugate across the tissue barrier to arrive at the target.

Embodiment 50. The use of Embodiment 48 or 49, wherein the tissue barrier is one of a blood-brain barrier, a mucosal barrier, a gastrointestinal barrier, a blood retina barrier (BRB), a blood-thymus barrier (BTB), a blood-testis barrier (BTB), and a blood-placenta barrier (BPB).

Embodiment 51. The use of Embodiment 46 or 47, wherein the target is a cell expressing a transferrin receptor, and the peptide-lipid-(lipid-based carrier) conjugate delivers the at least one reagent to the target by binding the peptide to the transferrin receptor expressed on the cell.

Embodiment 52. The use of Embodiment 51, wherein the transferrin receptor is located on a cell membrane of the cell, and binding of the peptide to the transferrin receptor induces transcytosis of the cells, thereby transporting the peptide-lipid-(lipid-based carrier) conjugate across the cell membrane to arrive into the cell.

Embodiment 53. The use of Embodiment 51 or 52, wherein the target is a cancer cell or a tissue cell.

Embodiment 54. The use of Embodiment 53, wherein the cancer is hepatocellular carcinoma, breast cancer, lung cancer, colon cancer, brain cancer, glioma, prostate cancer, ovarian cancer, or leukemia.

Embodiment 55. The use of Embodiment 53, wherein the tissue is skin, tonsil, tongue, oesophagus, cervix, kidney, placenta, pancreas, testis, anterior pituitary, stomach, breast, or liver.

Embodiment 56. The use of any one of Embodiments 46 to 55, wherein the target is in vivo or in vitro.

Embodiment 57. A peptide-lipid-(lipid-based carrier) conjugate for use in transporting at least one reagent across a tissue barrier of a subject, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating the at least one reagent;
   wherein a first carbon or a last carbon of the hydrophobic carbon chain is conjugated to a lysine of the peptide, and the hydrophobic carbon chain anchors into a lipid layer of the lipid-based carrier.

Embodiment 58. Use of a peptide-lipid-(lipid-based carrier) conjugate for the manufacture of a medicament for transporting at least one reagent across a tissue barrier of a subject, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating the at least one reagent;
   wherein a first carbon or a last carbon of the hydrophobic carbon chain is conjugated to a lysine of the peptide, and the hydrophobic carbon chain anchors into a lipid layer of the lipid-based carrier.

Embodiment 59. The use of Embodiment 57 or 58, wherein the tissue barrier is one of a blood-brain barrier, a mucosal barrier, a gastrointestinal barrier, a blood retina barrier (BRB), a blood-thymus barrier (BTB), a blood-testis barrier (BTB), and a blood-placenta barrier (BPB).

Embodiment 60. The use of any one of Embodiments 57 to 59, wherein one cell of the tissue barrier has a transferrin receptor.

Embodiment 61. The use of Embodiment 60, wherein the peptide binds to the transferrin receptor, and the binding of the peptide to the transferrin receptor induces transcytosis of the cells, thereby transporting the peptide-lipid-(lipid-based carrier) conjugate across the tissue barrier.

Embodiment 62. The use of any one of Embodiments 46 to 61, wherein the hydrophobic carbon chain comprises at least 6 carbons.

Embodiment 63. The use of any one of Embodiments 46 to 62, wherein the hydrophobic carbon chain is further conjugated to polyethylene glycol.

Embodiment 64. The use of any one of Embodiments 46 to 63, wherein the lipid-based carrier is selected form liposome, PEGylated liposome, cationic liposome, immunoliposome, transfersome, ethosome, niosome, virosome, nanoparticle, lipid nanoparticle, lipid nanoemulsion, microemulsion, pickering emulsion, micelle, cubosome, hexosome, spongosome, lipid-polymer hybrid nanoparticle, exosome, endosome, and extracellular vesicle.

Embodiment 65. The use of any one of Embodiments 46 to 64, wherein the at least one reagent is selected from siRNA, shRNA, microRNA, double-stranded RNA, single-stranded RNA, DNA, aptamers, peptides, proteins, small chemical molecules, large chemical molecules, viral particles or fragments thereof, dendrimers, ligands, eukaryotic cells or fragments thereof, and prokaryotic cells or fragments thereof.

Embodiment 66. A peptide-lipid-(lipid-based carrier) conjugate for use in the treatment and/or prevention of a central nervous system (CNS) disease, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating a therapeutic agent of the CNS disease;
   wherein a first carbon or a last carbon of the hydrophobic carbon chain is conjugated to a lysine of the peptide, and the hydrophobic carbon chain anchors into a lipid layer of the lipid-based carrier.

Embodiment 67. Use of a peptide-lipid-(lipid-based carrier) conjugate for the manufacture of a medicament for the treatment and/or prevention of a central nervous system (CNS) disease, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating a therapeutic agent of the CNS disease;
   wherein a first carbon or a last carbon of the hydrophobic carbon chain is conjugated to a lysine of the peptide, and the hydrophobic carbon chain anchors into a lipid layer of the lipid-based carrier.

Embodiment 68. The use of Embodiment 66 or 67, wherein the hydrophobic carbon chain comprises at least 6 carbons.

Embodiment 69. The use of any one of Embodiments 66 to 68, wherein the hydrophobic carbon chain is further conjugated to polyethylene glycol.

Embodiment 70. The use of any one of Embodiments 66 to 69, wherein the lipid-based carrier is selected form liposome, PEGylated liposome, cationic liposome, immunoliposome, transfersome, ethosome, niosome, virosome, nanoparticle, lipid nanoparticle, lipid nanoemulsion, microemulsion, pickering emulsion, micelle, cubosome, hexosome, spongosome, lipid-polymer hybrid nanoparticle, exosome, endosome, and extracellular vesicle.

Embodiment 71. The use of any one of Embodiments 66 or 70, wherein the peptide-lipid-(lipid-based carrier) conjugate is administered to a subject in need thereof.

Embodiment 72. The use of Embodiment 71, wherein the peptide binds to a transferrin receptor on a cell of a tissue barrier of the subject, and the binding of the peptide to the transferrin receptor induces transcytosis of the cells, thereby transporting the peptide-lipid-(lipid-based carrier) conjugate across the tissue barrier.

Embodiment 73. The use of Embodiment 72, wherein the tissue barrier is one of a blood-brain barrier, a mucosal barrier, a gastrointestinal barrier, a blood retina barrier (BRB), a blood-thymus barrier (BTB), a blood-testis barrier (BTB), and a blood-placenta barrier (BPB).

Embodiment 74. The use of any one of Embodiments 71 to 73, wherein the peptide-lipid-(lipid-based carrier) conjugate is administered to the subject in need thereof by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery.

Embodiment 75. The use of any one of Embodiments 66 to 74, wherein the CNS disease is one of Alzheimer's disease (AD), Parkinson's disease (PD), cerebrovascular accidents (CVA), ascular-related dementia, Creutzfeldt-Jakob disease (CJD), bovine spongiform encephalopathy (BSE), Traumatic Brain Injury (TBI), multiple sclerosis (MS), amyotrophic lateral sclerosis (ALS), Huntington's chorea, and spinal muscular atrophy (SMA).

Embodiment 76. The use of any one of Embodiments 71 to 75, wherein the subject is a mammal.

These and other aspects will become apparent from the following description of the preferred embodiment taken in conjunction with the following drawings.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows the schematic diagrams of the synthesis of the peptide-lipid conjugate disclosed in the present invention.

FIG. 2 shows the schematic diagrams of the synthesis of the peptide-lipid-(lipid-based carrier) conjugate disclosed in the present invention.

FIG. 3 shows the EC50 value of lipid nanoparticle (LNP) (negative control), VBT-034-Chol LNP, and VBT-034-PEG-Chol LNP obtained from Example 2 determined by fiber optic particle plasmon resonance (FOPPR) affinity study.

FIG. 4 shows the in vivo fluorescence imaging of mice after 0.25, 0.5, 1, 2, 4, and 6 hours after administration of the fluorescent tracer encapsulated LNP or VBT-034-Chol LNP obtained from Example 2 via intravenous (IV) injection.

FIG. 5 shows the quantification of fluorescence intensity in mouse brain of FIG. 4.

FIG. 6 shows the in vivo fluorescence imaging of mice after 0.25, 0.5, 1, 2, 4, 6, and 24 hours after oral administration of the fluorescent tracer encapsulated LNP or VBT-034-Chol LNP obtained from Example 2.

FIG. 7 shows the quantification of fluorescence intensity in mouse brain of FIG. 6.

### DETAILED DESCRIPTION OF THE PREFERRED EMBODIMENT

The present invention provides a peptide-lipid conjugate, comprising:
a peptide consisting of an amino acid sequence selected from the group consisting of SEQ ID NO: 1 to SEQ ID NO: 29; and
a hydrophobic carbon chain conjugated to the peptide.

Table 1 shows the peptide names and sequences of SEQ ID NO: 1 to SEQ ID NO: 29.

| SEQ ID NO | Peptide Name | Sequence |
|---|---|---|
| 1 | VBT-067 | DIHVLDKEYD |
| 2 | VBT-001 | DISVLMKEYD |
| 3 | VBT-004 | DIDVLMKEYD |
| 4 | VBT-008 | DIHVLMKEYD |
| 5 | VBT-011 | DILVLMKEYD |
| 6 | VBT-014 | DIPVLMKEYD |
| 7 | VBT-017 | DITVLMKEYD |
| 8 | VBT-022 | DISLLMKEYD |
| 9 | VBT-023 | DISVIMKEYD |
| 10 | VBT-024 | DISVVMKEYD |
| 11 | VBT-025 | DISVLDKEYD |
| 12 | VBT-026 | DISVLNKEYD |
| 13 | VBT-027 | DISVLCKEYD |
| 14 | VBT-028 | DISVLKKEYD |
| 15 | VBT-029 | DISVLEKEYD |
| 16 | VBT-030 | DISVLQKEYD |
| 17 | VBT-031 | DISVLMKEYN |
| 18 | VBT-033 | DISVLMKEYE |
| 19 | VBT-034 | DISVLMKEYQ |
| 20 | VBT-051 | DISVLSKEYD |
| 21 | VBT-054 | DISVLMKDYD |
| 22 | VBT-055 | DISVLMKEFD |
| 23 | VBT-056 | DISVLMKESD |
| 24 | VBT-057 | DISVLMKETD |
| 25 | VBT-062 | NISVLMKEYD |
| 26 | VBT-063 | DISELMKEYD |
| 27 | VBT-064 | DISVLLKEYD |
| 28 | VBT-065 | DISVLMKHYD |
| 29 | VBT-066 | DITVLCKEYN |

Preferably and in some embodiments, the peptides having the amino acid sequence of SEQ ID NO: 1 to SEQ ID NO: 29 have the binding affinity to a TfR. Additional details of these peptides can be found in US Patent publication no. 20240174716, the relevant disclosures of which are incorporated by reference herein for the subject matter and purpose referenced herein.

Preferably and in some embodiments, a first carbon or a last carbon of the hydrophobic carbon chain is conjugated to a lysine of the peptide. Preferably and in some embodiments, the first carbon or the last carbon of the hydrophobic carbon chain is conjugated to a lysine of the peptide via a triazole moiety.

Preferably and in some embodiments, the hydrophobic carbon chain comprises at least 6 carbons. Preferably and in some embodiments, the hydrophobic carbon chain is further conjugated to polyethylene glycol. Preferably and in some embodiments, the hydrophobic carbon chain is selected from dodecyne, cholesterol, and cholesterol-PEG.

Preferably and in some embodiments, the peptide-lipid conjugate disclosed herein further comprise a lipid-based carrier, and the hydrophobic carbon chain anchors into a lipid layer of the lipid-based carrier. Preferably and in some embodiments, at least one reagent is encapsulated in the lipid-based carrier.

The present invention also provides a peptide-lipid-(lipid-based carrier) conjugate, comprising:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating at least one reagent;
   wherein a first carbon or a last carbon of the hydrophobic carbon chain is conjugated to a lysine of the peptide, and the hydrophobic carbon chain anchors into a lipid layer of the lipid-based carrier.

Preferably and in some embodiments, the lipid-based carrier is selected form liposome, PEGylated liposome, cationic liposome, immunoliposome, transfersome, ethosome, niosome, virosome, nanoparticle, lipid nanoparticle, lipid nanoemulsion, microemulsion, pickering emulsion, micelle, cubosome, hexosome, spongosome, lipid-polymer hybrid nanoparticle, exosome, endosome, and extracellular vesicle.

Preferably and in some embodiments, the at least one reagent is selected from siRNA, shRNA, microRNA, double-stranded RNA, single-stranded RNA, DNA, aptamers, peptides, proteins, small chemical molecules, large chemical molecules, viral particles or fragments thereof, dendrimers, ligands, eukaryotic cells or fragments thereof, and prokaryotic cells or fragments thereof.

As used herein, the term “hydrophobic carbon chain” refers to a hydrocarbon group having a carbon backbone that is substantially insoluble in water and that increases the hydrophobicity (lipophilicity) of the molecule to which it is attached. Preferably and in some embodiments, the hydrophobic carbon chain included in the peptide-lipid conjugate/peptide-lipid-(lipid-based carrier) conjugate comprises at least 6 carbons. Preferably and in some embodiments, the hydrophobic carbon chain comprises from 6 to about 30 carbons. The hydrophobic carbon chain may be straight-chain or branched, may be saturated or unsaturated (e.g., containing one or more double or triple bonds), and may be acyclic and/or cyclic. Preferably and in some embodiments, the hydrophobic carbon chain is an alkyl, alkenyl, alkynyl, cycloalkyl, cycloalkylalkyl, arylalkyl, or alkylaryl group. The hydrophobic carbon chain may optionally bear one or more non-interfering substituents that do not substantially increase the overall hydrophilicity of the group (for example, halogen, hydroxy, alkoxy, or amino substituents), provided that the group as a whole remains hydrophobic. Examples of the hydrophobic carbon chain include, but are not limited to, n-hexyl, isohexyl, 3-methylpentyl, cyclohexyl, n-heptyl, isoheptyl, 3-methylhexyl, cycloheptyl, n-octyl, iso-octyl, 2-ethylhexyl, n-nonyl, isononyl, n-decyl, isodecyl, 2-propylheptyl, n-undecyl, iso-undecyl, n-dodecyl, lauryl, isododecyl, n-tridecyl, isotridecyl, n-tetradecyl, myristyl, isotetradecyl, n-pentadecyl, isopentadecyl, n-hexadecyl, cetyl, palmitoyl, n-heptadecyl, isoheptadecyl, n-octadecyl, stearyl, oleyl (unsaturated), n-nonadecyl, n-eicosyl, arachidyl, n-heneicosyl, n-docosyl, behenyl, n-tricosyl, n-tetracosyl, lignoceryl, n-pentacosyl, n-hexacosyl, cerotyl, n-heptacosyl, n-octacosyl, montanyl, n-nonacosyl, n-triacontyl, 2-methyldodecyl, 3-methyltetradecyl, 2-ethylhexadecyl, dodecenyl, tetradecenyl, hexadecenyl, octadecenyl, octadecadienyl, octadecatrienyl, dodecylbenzyl, tetradecylbenzyl, dodecylphenethyl, and cholesterol.

As used herein, the term “lipid-based carrier” refers to a delivery vehicle that comprises one or more lipid components and that is configured to encapsulate, solubilize, adsorb, or otherwise physically or chemically associate with an active agent (e.g., a therapeutic, diagnostic, prophylactic, or cosmetic agent) and facilitate its storage, handling, and/or delivery to a subject or to a target site. A lipid-based carrier typically comprises a lipid phase that forms a continuous or discontinuous phase surrounding, enclosing, or interspersed with the active agent. The lipid component may include, for example, phospholipids, glycerides (mono-, di- and triglycerides), fatty acids, fatty alcohols, sterols (e.g., cholesterol), sphingolipids, glycolipids, synthetic cationic or ionizable lipids, PEG-lipids, and mixtures thereof, which may be of natural, semi-synthetic, or fully synthetic origin. In certain embodiments, the lipid-based carrier is present in the form of particles, vesicles, micelles, droplets, or films, having a size ranging from the nanometer to the millimeter scale. The lipid-based carrier may be formulated as an aqueous dispersion, suspension, emulsion, gel, or lyophilized (freeze-dried) solid, and may optionally further comprise one or more non-lipid excipients, such as surfactants, stabilizers, cryoprotectants, tonicity agents, buffers, or polymers.

Examples of lipid-based carriers include, but are not limited to liposomes and vesicles (such as multilamellar vesicles (MLVs), small unilamellar vesicles (SUVs), large unilamellar vesicles (LUVs), conventional, cationic, anionic, zwitterionic, or PEGylated liposomes, pH-sensitive or fusogenic liposomes), lipid nanoparticles (LNPs) (such as ionizable cationic lipid nanoparticles for delivery of nucleic acids (e.g., mRNA, siRNA), cationic lipid nanoparticles for DNA or RNA delivery, lipid-polymer hybrid nanoparticles having a lipid shell and a polymeric core), solid lipid nanoparticles (SLNs) and nanostructured lipid carriers (NLCs) (such as nanoparticles comprising a solid lipid core (e.g., triglycerides, fatty acids, waxes) stabilized by surfactants and nanostructured lipid carriers comprising mixtures of solid and liquid lipids), lipid micelles and mixed micelles (such as micelles formed from phospholipids, bile salt derivatives, or synthetic surfactant-lipid combinations, mixed micelles comprising phospholipids and non-ionic surfactants for solubilization of poorly water-soluble drugs), emulsions and related systems (such as oil-in-water (O/W) or water-in-oil (W/O) emulsions in which the oil phase comprises one or more lipids, multiple emulsions (e.g., W/O/W emulsions), microemulsions or nanoemulsions with a lipid oil phase and optional co-surfactants), lipid-coated or lipid-shell particles (such as lipid-coated inorganic or polymeric nanoparticles (e.g., silica, gold, PLGA), lipid-core/lipid-shell structures (lipospheres, lipid microspheres)), lipid-drug conjugates and lipoprotein-mimetic systems (such as prodrugs in which the active agent is covalently linked to a lipid moiety (e.g., fatty acid, phospholipid) to form a self-assembling lipidic carrier, lipoprotein-like particles (e.g., high-density lipoprotein (HDL) mimetics) comprising a lipid core and an apolipoprotein or peptide shell), and hybrid/biological lipid carriers (such as lipid-polymer hybrid nanoparticles, exosomes/extracellular vesicles, cell-membrane-coated lipid nanoparticles).

As used herein, the term “peptide” refers to a molecular chain of amino acids, including both L-forms and D-forms. The amino acids, if required, can be modified in vivo or in vitro, for example by manosylation, glycosylation, amidation (specifically C-terminal amides), carboxylation or phosphorylation with the stipulation that these modifications must preserve the biological activity of the original molecule. In addition, peptides can be part of a chimeric protein.

Functional derivatives of the peptides are also included in the present invention. Functional derivatives are meant to include peptides which differ in one or more amino acids in the overall sequence, which have deletions, substitutions, inversions or additions. Amino acid substitutions which can be expected not to essentially alter biological and immunological activities have been described. Amino acid replacements between related amino acids or replacements which have occurred frequently in evolution include, inter alia Ser/Ala, Ser/Gly, Asp/Gly, Asp/Asn and Ile/Val.

The peptides according to the invention can be produced synthetically or by recombinant DNA technology. Methods for producing synthetic peptides are well known in the art.

As used herein, the nomenclature used to describe peptides of the present invention follows the conventional practice in which the amino group (N-terminus) and/or the 5′ are presented to the left and the carboxyl group (C-terminus) and/or 3′ are presented to the right.

As used herein, the term “transferrin receptor (TfR)” refers to a type II transmembrane glycoprotein having a molecular weight of 90 kDa and found forming a homodimer (180 kDa) linked by disulfide bond on the surface of the cell on which it located. The amino acid sequence of human TfR is as shown as the protein number P02786 on the Uniprot website (https://www.uniprot.org/uniprotkb/P02786/entry). Each extracellular domain of TfR is consisted of three domains including apical domain (189th to 383rd amino acid residues), protease like domain (122nd to 188th amino acid residues and 384th to 606th amino acid residues), and helical domain (607th to 760th amino acid residues). The primary function of TfR is to bind to transferrin (Tf), a protein that carries iron in the bloodstream, and facilitate the uptake of iron into cells. The transferrin binding region is major on the TfR1 dimer surface of the helical domain and a small part of protease-like domain. The binding of TfR to transferrin induces transcytosis of the cell on which the TfR is located, thereby transporting the Tf and the iron carried by the Tf into the cell. Transferrin receptors are commonly found on various tissue barriers, including blood-brain barrier, gastrointestinal barrier, mucosal barrier, a blood retina barrier (BRB), a blood-thymus barrier (BTB), a blood-testis barrier (BTB), and a blood-placenta barrier (BPB). In addition, transferrin receptors have been found to have relatively high expression levels in rapidly proliferating cells (e.g., cancer cells), such as those associated with hepatocellular carcinoma, breast cancer, lung cancer, colon cancer, brain cancer, glioma, prostate cancer, ovarian cancer, or leukemia. Transferrin receptors have also been found to be expressed in cells of some tissues, including, but not limited to, skin, tonsil, tongue, oesophagus, cervix, kidney, placenta, pancreas, testis, anterior pituitary, stomach, breast, or liver.

The present invention also provides a method of transporting at least one reagent to a target, comprising binding the peptide-lipid-(lipid-based carrier) conjugate disclosed herein to a transferrin receptor. The present invention also provides the peptide-lipid-(lipid-based carrier) conjugate disclosed herein for use in delivering at least one reagent to a target. The present invention also provides use of the peptide-lipid-(lipid-based carrier) conjugate disclosed herein for the manufacture of a medicament for delivering at least one reagent to a target. The target is in vivo or in vitro.

Preferably and in some embodiments, the at least one reagent has to be transported across a tissue barrier to arrive at the target, and the transferrin receptor is located on a cell of the tissue barrier. Preferably and in some embodiments, binding of the peptide to the transferrin receptor induces transcytosis of the cells, thereby transporting the peptide-lipid-(lipid-based carrier) conjugate across the tissue barrier to arrive at the target. Preferably and in some embodiments, the tissue barrier is one of a blood-brain barrier (BBB), a mucosal barrier, a gastrointestinal barrier, a blood retina barrier (BRB), a blood-thymus barrier (BTB), a blood-testis barrier (BTB), and a blood-placenta barrier (BPB). Preferably and in some embodiments, the target is a brain cell. More preferably and in some preferred embodiments, the tissue barrier is blood-brain barrier, and the target is a brain cell.

Preferably and in some embodiments, the target is a cell expressing the transferrin receptor, the transferrin receptor is located on a cell membrane of the cell, and binding of the peptide-lipid-(lipid-based carrier) conjugate to the transferrin receptor induces transcytosis of the cells, thereby transporting the peptide-lipid-(lipid-based carrier) conjugate across the cell membrane to arrive into the cell. Preferably and in some embodiments, the target is a cancer cell. More preferably and in some preferred embodiments, the cancer is hepatocellular carcinoma, breast cancer, lung cancer, colon cancer, brain cancer, glioma, prostate cancer, ovarian cancer, or leukemia. Preferably and in some embodiments, the target is a tissue cell. More preferably and in some preferred embodiments, the tissue is skin, tonsil, tongue, oesophagus, cervix, kidney, placenta, pancreas, testis, anterior pituitary, stomach, breast, or liver.

The present invention also provides a method of transporting at least one reagent across a tissue barrier of a subject, comprising administering to the subject the peptide-lipid-(lipid-based carrier) conjugate disclosed herein. The present invention also provides the peptide-lipid-(lipid-based carrier) conjugate disclosed herein for use in transporting at least one reagent across a tissue barrier of a subject. The present invention also provides use of the peptide-lipid-(lipid-based carrier) conjugate disclosed herein for the manufacture of a medicament for transporting at least one reagent across a tissue barrier of a subject.

Preferably and in some embodiments, the tissue barrier is one of a blood-brain barrier, a mucosal barrier, a gastrointestinal barrier, a blood retina barrier (BRB), a blood-thymus barrier (BTB), a blood-testis barrier (BTB), and a blood-placenta barrier (BPB). Preferably and in some embodiments, cells of the tissue barrier express transferrin receptors. Preferably and in some embodiments, the peptide binds to the transferrin receptor, and the binding of the peptide to the transferrin receptor induces transcytosis of the cells, thereby transporting the peptide-lipid-(lipid-based carrier) conjugate disclosed herein across the tissue barrier. Preferably and in some embodiments, the peptide-lipid-(lipid-based carrier) conjugate is administered to the subject by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery. Preferably and in some embodiments, the subject is a mammal. More preferably and in some preferred embodiments, the subject is a rodent or a human.

The present invention also provides a method of treating or preventing a central nervous system (CNS) disease, comprising administering a subject in need thereof a pharmaceutically effective amount of the peptide-lipid-(lipid-based carrier) conjugate disclosed herein. The present invention also provides the peptide-lipid-(lipid-based carrier) conjugate disclosed herein for use in the treatment and/or prevention of a central nervous system (CNS) disease. The present invention also provides use of the peptide-lipid-(lipid-based carrier) conjugate disclosed herein for the manufacture of a medicament for the treatment and/or prevention of a central nervous system (CNS) disease.

Preferably and in some embodiments, the at least one agent is a therapeutic agent of the CNS disease. Preferably and in some embodiments, the peptide binds to a transferrin receptor on a cell of a tissue barrier of the subject, and the binding of the peptide to the transferrin receptor induces transcytosis of the cell, thereby transporting the peptide-lipid-(lipid-based carrier) conjugate across the tissue barrier. Preferably and in some embodiments, the tissue barrier is one of a blood-brain barrier, a mucosal barrier, a gastrointestinal barrier, a blood retina barrier (BRB), a blood-thymus barrier (BTB), a blood-testis barrier (BTB), and a blood-placenta barrier (BPB). Preferably and in some embodiments, the subject is a mammal. More preferably and in some preferred embodiments, the subject is a rodent or a human. Preferably and in some embodiments, the peptide-lipid-(lipid-based carrier) conjugate is administered to the subject in need thereof by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery. Preferably and in some embodiments, the CNS disease is one of Alzheimer's disease (AD), Parkinson's disease (PD), cerebrovascular accidents (CVA), ascular-related dementia, Creutzfeldt-Jakob disease (CJD), bovine spongiform encephalopathy (BSE), Traumatic Brain Injury (TBI), multiple sclerosis (MS), amyotrophic lateral sclerosis (ALS), Huntington's chorea, and spinal muscular atrophy (SMA).

Formulations suitable for administration of the present invention may comprise, possibly among other things well known to those of skill in the art: aqueous and non-aqueous solutions, antioxidants, bacteriostats, buffers, solutes that affect isotonicity, preservatives, solubilizers, stabilizers, suspending agents, thickening agents, or a combination thereof.

In addition or in the alternative, formulations suitable for administration of the present invention may comprise, possibly among other things well known to those of skill in the art: gels, PEG such as PEG 400, propylene glycol, saline, sachets, water, other appropriate liquids known in the art, or a combination thereof.

Also in the addition or in the alternative, formulations suitable for administration of the present invention may comprise, possibly among other things well known to those of skill in the art: binders, buffering agents, calcium phosphates, cellulose, colloids, such as colloidal silicon dioxide, colorants, diluents, disintegrating agents, dyes, fillers, flavoring agents, gelatin, lactose, magnesium stearate, mannitol, microcrystalline gelatin, moistening agents, paraffin hydrocarbons, pastilles, polyethylene glycols, preservatives, sorbitol, starch, such as corn starch, potato starch, or a combination thereof, stearic acid, sucrose, talc, triglycerides, or a combination thereof.

Also in addition or in the alternative, formulations suitable for administration of the present invention may comprise, possibly among other things well known to those of skill in the art: alcohol such as benzyl alcohol or ethanol, benzalkonium chloride, buffers such as phosphate buffers, acetate buffers, citrate buffers, or a combination thereof, carboxymethylcellulose or microcrystalline cellulose, cholesterol, dextrose, juice such as grapefruit juice, milk, phospholipids such as lecithin, oil such as vegetable, fish, or mineral oil, or a combination thereof, other pharmaceutically compatible carriers known in the art, or a combination thereof.

Also in the addition or in the alternative, formulations suitable for administration of the present invention may comprise, possibly among other things well known to those of skill in the art: biodegradables such as poly-lactic-coglycolic acid (PLGA) polymer, other entities whose degradation products can quickly be cleared from a biological system, or a combination thereof.

Formulations of the present invention may be administered in unit-dose form, multi-dose form, or a combination thereof. They may be packaged in unit-dose containers, multi-dose containers, or a combination thereof. The present invention may exist in ampoules, cachets, capsules, granules, lozenges, powders, tablets, vials, emulsions, including but not limited to acacia emulsions, suspensions, or a combination thereof.

As used herein, an “effective amount” or a “sufficient amount” of a substance is that amount sufficient to effect beneficial or desired results, including clinical results, and, as such, an “effective amount” depends upon the context in which it is being applied. In the context of administering an immunogenic composition, the effective amount is an immunogenically effective amount, which contains sufficient immunogenic composition of the present invention to elicit an immune response. In the context of administering a pharmaceutical composition, the effective amount is a pharmaceutically effective amount, which contains sufficient pharmaceutical composition of the present invention to maintain or produce a desired physiological result. An effective amount can be administered in one or more doses.

As used herein, the term “pharmaceutically effective amount” refers to an amount capable of or sufficient to maintain or produce a desired physiological result, including but not limited to treating, reducing, attenuating, eliminating, suppressing, substantially preventing, or prophylaxing, or a combination thereof, a disease, disorder, or combination thereof. A pharmaceutically effective amount may comprise one or more doses administered sequentially or simultaneously. Those skilled in the art will know to adjust doses of the present invention to account for various types of formulations, including but not limited to slow-release formulation. As used herein, the term “prophylactic” refers to a composition capable of substantially preventing or prophylaxing any aspect of a disease, disorder, or combination thereof. As used herein, the term “therapeutic” refers to a composition capable of treating, reducing, halting the progression of, slowing the progression of, beneficially altering, eliminating, or a combination thereof, any aspect of a disease, disorder, or combination thereof.

The term “dose” as used herein in reference to a composition refers to a measured portion of the composition taken by (administered to or received by) a subject at any one time.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In some embodiments, the subject is a human. Human subjects may also include fetuses.

As used herein, the terms “subject,” refers to any subject, particularly a mammalian subject, for whom therapy is desired, for example, a human.

The term “treat,” “treating,” or “treatment” as used herein encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevent,” “preventing,” or “prevention” refers to being able to substantially preclude, avert, obviate, forestall, stop, hinder, or a combination thereof, any aspect of a disease, condition, or combination thereof from happening, especially by advance action.

In some embodiments, the oligonucleotide therapeutics and/or the composition of the present invention may be administered to subjects by a variety of administration modes, including by intradermal, intramuscular, subcutaneous, intravenous, intra-atrial, intra-articular, intraperitoneal, parenteral, oral, rectal, intranasal, intrapulmonary, and transdermal delivery, or topically to the eyes, ears, skin or mucous membranes. Alternatively, the antigen may be administered ex-vivo by direct exposure to cells, tissues or organs originating from a subject (autologous) or another subject (allogeneic), optionally in a biologically suitable, liquid or solid carrier.

The meaning of the technical and scientific terms as described herein can be clearly understood by a person of ordinary skill in the art.

As used herein, the term “about,” “around,” or “approximately” when combined with a value refers to plus and minus 10% of the reference value. For example, a length of about 1000 nanometers (nm) refers to a length of the range of 900 nm to 1100 nm.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### Example 1 Preparation of Peptide-Lipid Conjugates

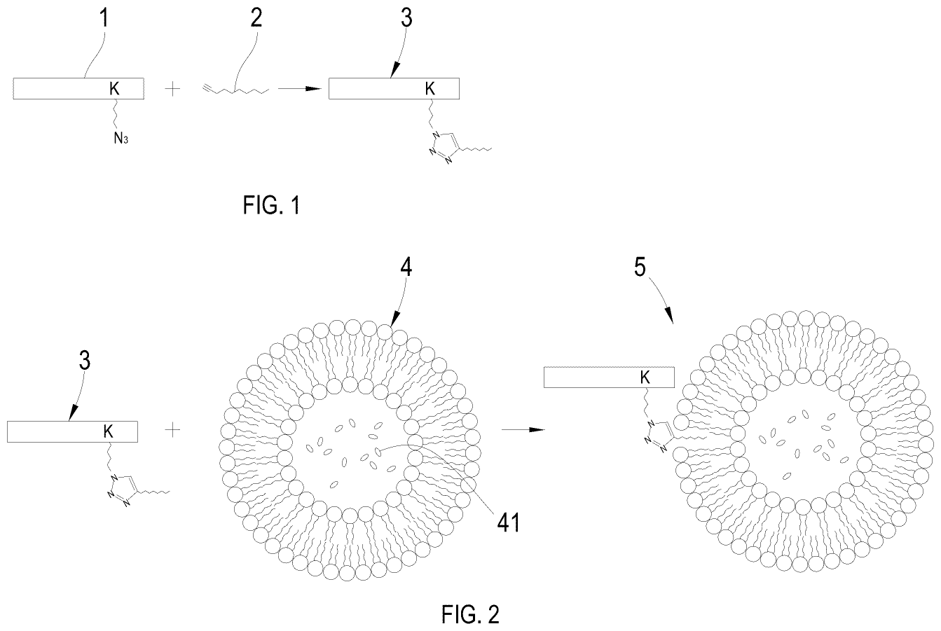

As shown in FIG. 1, peptide-lipid conjugates 3 described herein was synthesized by conjugating an azide peptide 1 having the amino acid sequence of one of SEQ ID NO: 1 to SEQ ID NO: 29 to a hydrophobic carbon chain 2 (such as 1-dodecyne, alkyne cholesterol, and cholesterol-PEG-DBCO) in this Example.

### Materials and Methods

To synthesize peptide-dodecyne conjugates, Solution A, containing 40 μL of 100 mM THPTA (tris-hydroxypropyltriazolylmethylamine) and 20 μL of 100 mM CuSO₄, and Solution B, containing 20 μL of 1-dodecyne and 480 μL dimethyl sulfoxide (DMSO), were mixed completely. After that, Solution C, containing 375 μL of H₂O and 25 μL of 8 mM azide VBT-001 (SEQ ID NO: 2) or azide VBT-025 (SEQ ID NO: 11) or azide VBT-034 (SEQ ID NO: 19), was added into the mixture and mixed completely. Then, 40 μL of 100 mM ascorbic acid was added to the mixture and incubated for 6 hours at room temperature. The reaction resultants (VBT-001-C12, VBT-025-C12, VBT-034-C12) were subject to liquid chromatography/mass spectrometry (LC/MS) analysis.

To synthesize peptide-cholesterol conjugates, Solution A, containing 40 μL of 100 mM THPTA and 20 μL of 100 mM CuSO₄, and Solution B, containing 20 μL of alkyne cholesterol and 480 μl DMSO, were mixed completely. After that, Solution C, containing 375 μL of H₂O and 25 μL of 8 mM azide VBT-034 (SEQ ID NO: 19), was added into the mixture and mixed completely. Then, 40 μL of 100 mM ascorbic acid was added to the mixture and incubated for 6 hours at room temperature. The reaction resultant (VBT-034-Chol) was subject to LC/MS analysis.

To synthesize peptide-PEG cholesterol conjugates, Solution A, containing 26 μL of cholesterol-polyethylene glycol (PEGₙ, n=8-14)-dibenzocyclooctyne (DBCO) and 449 μL of DMSO, and Solution B, containing 25 μL of 8 mM azide VBT-034 (SEQ ID NO: 19), were mixed completely and then incubated for 6 hours at room temperature. The reaction resultant (VBT-034-PEG-Chol) was subject to LC/MS analysis.

### Results

LC/MS analysis showed that the synthetic VBT-001-C12, VBT-025-C12, VBT-034-C12, VBT-034-Chol, and VBT-034-PEG-Chol had observed molecular weights of around 1404.5 g/mole, 1388.38 g/mole, 1418.25 g/mole, 1647.81 g/mole, and 2392-2656 g/mole, respectively. These values are consistent with the corresponding theoretical molecular weights, indicating that the conjugates were correctly synthesized.

### Example 2 Preparation of Peptide-Lipid-(Lipid-Based Carrier) Conjugates

As shown in FIG. 2, peptide-lipid-(lipid-based carrier) conjugates 5 described herein was synthesized by combining the peptide-lipid conjugate 3 obtained in Example 1 with a lipid-based carrier 4 encapsulating at least one reagent 41 in this Example. More specifically, the peptide-lipid-(lipid-based carrier) conjugates 5 was formed by anchoring the hydrophobic carbon chain of the peptide-lipid conjugate 3 into a lipid layer of the lipid-based carrier 4.

### Materials and Methods

In this Example, the lipide-based carrier is lipid nanoparticles (LNP). Peptide-Lipid-LNP conjugates were manufactured by a microfluidic device (NanoAssemblr™ Ignite, PNI, Cytiva, Marlborough, MA, USA). To produce VBT-034-Chol LNP, oil phase was first produced by mixing 80 μL of VBT-034-Chol obtained from Example 1 with 21 μL of 100 mg/mL 1-Octylnonyl 8-[(2-hydroxyethyl)[6-oxo-6-(undecyloxy)hexyl]amino]octanoate (SM-102), 19.2 μL of 25 mg/mL 1,2-Distearoyl-sn-glycero-3-phosphocholine (1,2-DSPC), 177.6 μL of 5 mg/mL cholesterol, 226.8 μL of 1 mg/mL DMG-PEG (2000), and absolute ethanol to a final volume of 0.6 mL. Aqueous phase was produced by diluting VivoTag 680XL dye with 1× phosphate-buffered saline (PBS) buffer to a concentration of 60 μg/mL for intravenous (IV) injection study or to a concentration of 30 μg/mL for oral delivery study. A total of 1.6 mL of VBT-034-Chol LNP was produced by the microfluidic device (NanoAssemblr™ Ignite) at a ratio of 3:1 (aqueous phase:oil phase) at a flow rate of 8 ml/min. The obtained VBT-034-Chol LNP was then subject to dynamic light scattering (DLS) analysis to measure the diameter of the conjugate.

To produce VBT-034-PEG-Chol LNP, oil phase was first produced by mixing 75 μL of VBT-034-PEG-Chol obtained from Example 1 with 17.5 μL of 100 mg/mL SM-102, 16 μL of 25 mg/mL 1,2-DSPC, 148 μL of 5 mg/mL cholesterol, 189 μL of 1 mg/mL DMG-PEG (2000), and absolute ethanol to a final volume of 0.5 mL. Aqueous phase was produced by diluting VivoTag 680XL dye with 1×PBS buffer to a concentration of 60 μg/mL for i.v. injection study. A total of 1.2 mL of VBT-034-PEG-Chol LNP was produced by the microfluidic device (NanoAssemblr™ Ignite) at a ratio of 3:1 (aqueous phase:oil phase) at a flow rate of 8 ml/min. The obtained VBT-034-PEG-Chol LNP was then subject to DLS analysis to measure the diameter of the conjugate.

To produce LNP as a negative control, oil phase was first produced by mixing 21 μL of 100 mg/mL 1-Octylnonyl 8-[(2-hydroxyethyl)[6-oxo-6-(undecyloxy)hexyl]amino]octanoate (SM-102), 19.2 μL of 25 mg/mL 1,2-Distearoyl-sn-glycero-3-phosphocholine (1,2-DSPC), 177.6 μL of 5 mg/mL cholesterol, 226.8 μL of 1 mg/mL DMG-PEG (2000), and absolute ethanol to a final volume of 0.6 mL. Aqueous phase was produced by diluting VivoTag 680XL dye with Ix phosphate-buffered saline (PBS) buffer to a concentration of 60 μg/mL for IV injection study or to a concentration of 30 μg/mL for oral delivery study. A total of 1.6 mL of LNP was produced by the microfluidic device (NanoAssemblr™ Ignite) at a ratio of 3:1 (aqueous phase:oil phase) at a flow rate of 8 ml/min. The obtained LNP was then subject to dynamic light scattering (DLS) analysis to measure the diameter of the conjugate.

### Results

DLS analysis showed that the average diameters of VBT-034-Chol LNP, VBT-034-PEG-Chol LNP, and LNP were around 297.7 nm, around 85.08 nm, and around 49.05 nm, respectively.

### Example 3 TfR Binding Assay of Peptide-Lipid-(Lipid-Based Carrier) Conjugates

### Materials and Methods

To determine the affinity of the peptide-lipid-(lipid-based carrier) conjugates to TfR1, fiber optic particle plasmon resonance (FOPPR) affinity study was performed according to the manufacturer's instruction. Briefly, 80 μL of TfR1 (10 μg/ml) (Acro Biosystems. Cat. No. CD1-H5243, Newark, DE, USA) dissolved in PBS buffer (pH 7.4) was pipetted into the EDC/NHS preactivated surface of standard chip NanoAu-MM (Taipei, Taiwan) for protein conjugating to the carboxyl group of the surface sensor chip. VBT-034-Chol LNP and VBT-034-PEG-Chol LNP obtained from Example 2 and LNP (which serves as the negative control) were loaded onto the chip with different 3-fold serial dilution starting from 1× to 1/243×, respectively. Based on the change of signal ration, the reciprocal of the highest dilution capable of reaching 50% of maximal effect (EC₅₀) of binding activity was calculated and defined as the EC₅₀ value.

### Results

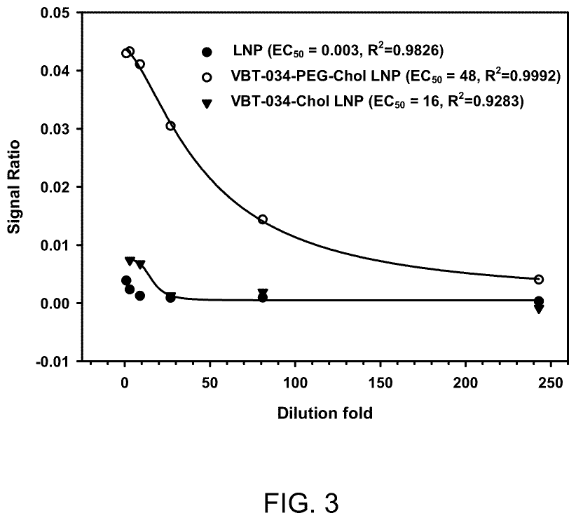

The affinity of the peptide-lipid-LNP conjugates disclosed herein to the transferrin receptor is expressed by EC₅₀ value. The higher the EC₅₀ value (which means the higher dilution fold), the stronger the binding between the two molecules. As shown in FIG. 3, the EC₅₀ value of LNP, which serves as the negative control, was less than 1, indicating that the LNP did not bind to TfR1. In contrast, the EC₅₀ values of VBT-034-PEG-Chol LNP and VBT-034-Chol LNP were 48 and 16, respectively, indicating that each of 48-fold dilution of VBT-034-PEG-Chol LNP and 16-fold dilution of VBT-034-Chol LNP reached 50% of maximal effect.

### Example 4. Biodistribution of Peptide-Lipid-(Lipid-Based Carrier) Conjugates by IV Injection in Mice

### Materials and Methods

BALB/c mice (BioLASCO Taiwan Co., Ltd, Taipei, Taiwan) at the age of 6-8 weeks were divided into a control group (n=2) and a test group (n=2). Mice in the control group were administered 50 μL of the fluorescent tracer encapsulated LNP via intravenous (IV) injection. Mice in the test group were administered 50 μL of the fluorescent tracer encapsulated VBT-034-Chol LNP obtained from Example 2 via intravenous (IV) injection. Mice were photographed using an optical imaging system (U-OI, MiLabs, Netherlands) at 0.25, 0.5, 1, 2, 4, and 6 hours after the injection for an in vivo fluorescence imaging system (IVIS) imaging analysis, with a condition of the excitation wavelength of 655 nm, the emission wavelength of 710 nm, 10-second exposure time, and f/4.0. Fluorescence signals of mouse brains were further quantified.

### Results

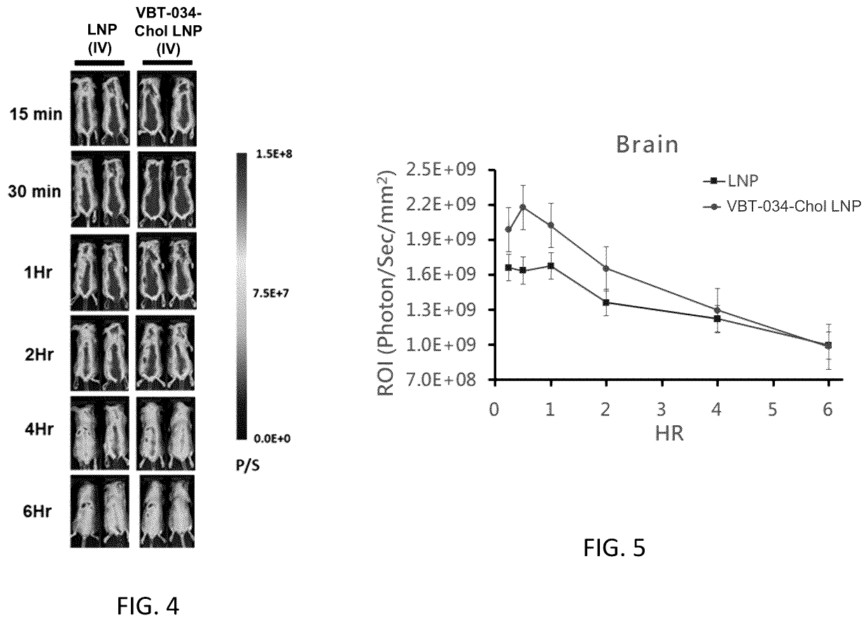

As shown in FIG. 4, as soon as 0.25 hours (15 minutes) after injection, the fluorescent signal was enriched in the brain and spine of the mice in the test group. The fluorescent signal was also observed in the control group, due to non-specific hydrophobic binding of the high dose of the LNP to lipid-enriched sphingomyelin as a background. Then, fluorescence signals of mouse brains were further quantified. As shown in FIG. 5, compared with the control group, a higher level of fluorescent signal was observed in the brains of mice in the test group after 0.25 hours (15 minutes) of the injection. The pharmacokinetic exposure of the conjugate within the brain was quantified by calculating the area under the curve (AUC) of fluorescent intensity from 15 minutes to 6 hours (AUC₁₅ₘᵢₙ₋₆ₕᵣₛ). Following IV injection, the control LNP exhibited a brain AUC₁₅ₘᵢₙ₋₆ₕᵣₛ of 7.55×10⁹ photons/s/mm²·hrs. In contrast, the VBT-034-Chol LNP formulation demonstrated an improved brain AUC₁₅ₘᵢₙ₋₆ₕᵣₛ of 8.64×10⁹ photons/s/mm²·hrs. This represents a 14.4% increase in systemic brain exposure for the VBT-034-Chol LNP compared to the control LNP group. The results prove that the peptide-lipid-LNP conjugates disclosed herein are more effective in targeting the brain and spine than LNP alone and can be used as an effective drug delivery system.

### Example 5. Biodistribution of Peptide-Lipid-(Lipid-Based Carrier) Conjugates by Oral Administration in Mice

### Materials and Methods

BALB/c mice (BioLASCO Taiwan Co., Ltd, Taipei, Taiwan) at the age of 6-8 weeks were divided into a control group (n=2) and a test group (n=2). Mice in the control group were administered 50 μL of the fluorescent tracer encapsulated LNP via oral administration. Mice in the test group were administered 50 μL of the fluorescent tracer encapsulated VBT-034-Chol LNP obtained from Example 2 via oral administration. Mice were photographed using an optical imaging system (U-OI, MiLabs, Netherlands) at 0.25, 0.5, 1, 2, 4, 6, and 24 hours after the injection for an IVIS imaging analysis, with a condition of the excitation wavelength of 655 nm, the emission wavelength of 710 nm, 10-second exposure time, and f/4.0. Fluorescence signals of mouse brains were further quantified.

### Results

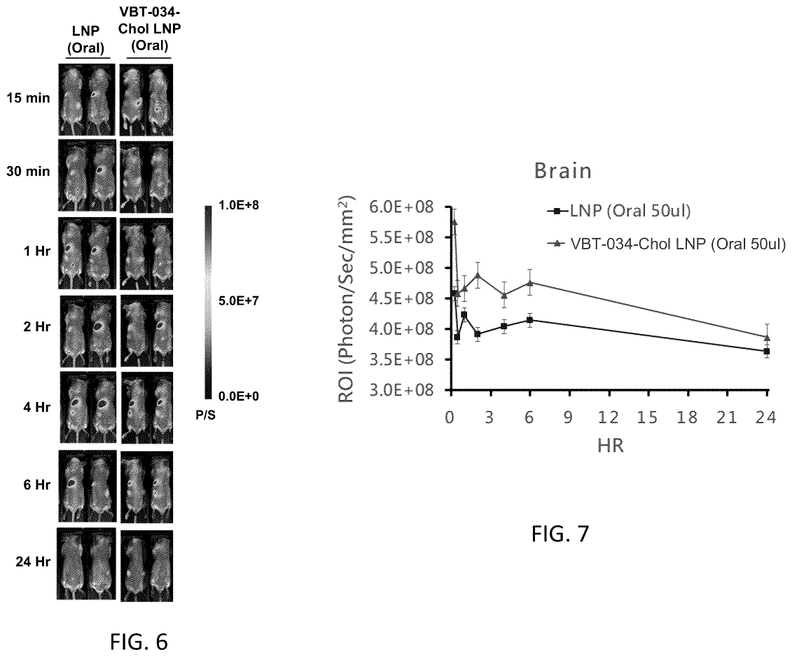

As shown in FIG. 6, as soon as 0.25 hours (15 minutes) after oral administration, the fluorescent signal was enriched in the brain and liver of the mice in the test group. The fluorescent signal was also observed in metabolic organs, such as liver and kidneys, in the control group as a background. Then, fluorescence signals of mouse brains were further quantified. As shown in FIG. 7, compared with the control group, a higher level of fluorescent signal was observed in the brains of mice in the test group after 0.25 hours (15 minutes) of the oral administration. The pharmacokinetic exposure of the conjugate within the brain was quantified by calculating the AUC of fluorescent intensity from 15 minutes to 6 hours (AUC₁₅ₘᵢₙ₋₆ₕᵣₛ). Following oral administration, the control LNP exhibited a brain AUC₁₅ₘᵢₙ₋₆ₕᵣₛ of 2.33×10⁹ photons/s/mm²·hrs. In contrast, the VBT-034-Chol LNP formulation demonstrated an improved brain AUC₁₅ₘᵢₙ₋₆ₕᵣₛ of 2.72×10⁹ photons/s/mm²·hrs. This represents a 17% increase in systemic brain exposure for the VBT-034-Chol LNP compared to the control LNP group. The results prove that the peptide-lipid-LNP conjugates disclosed herein are more effective in targeting the brain than LNP alone after oral administration and can be used as an effective drug delivery system.

The results indicate that the peptide-lipid-(lipid-based carrier) conjugates disclosed herein not only cross the blood-brain barrier but also quickly distribute in the brain within 15-30 minutes via both intravenous injection or oral administration. Therefore, then peptide-lipid-(lipid-based carrier) conjugates disclosed herein overcome the therapeutic limitations of LNP caused by the liver clearance rate.

Oral administration of the peptide-lipid-(lipid-based carrier) conjugates disclosed herein allows the peptide-lipid-(lipid-based carrier) conjugates bind to the TfR on the cells of the gastrointestinal mucosal barrier, and the binding of the peptide-lipid-(lipid-based carrier) conjugates and the TfR induces transcytosis of the cells, thereby transporting the peptide-lipid-(lipid-based carrier) conjugates across the gastrointestinal mucosal barrier to enter the body and further arrive at the brain. This peptide-lipid-(lipid-based carrier) conjugates can also be transported across the blood-brain barrier with the same mechanism.

To sum up, the Examples disclosed herein prove that the peptide-lipid-(lipid-based carrier) conjugates of the present invention have binding affinity to TfR. The binding of the peptide-lipid-(lipid-based carrier) conjugates to TfRs on cells of a tissue barrier induces transcytosis of the cells, thereby transporting the peptide-lipid-(lipid-based carrier) conjugates across the gastrointestinal mucosal barrier to enter the body and further arrive at the brain. The peptide-lipid-(lipid-based carrier) conjugates can deliver reagents encapsulated in the lipid-based carrier across a tissue barrier and effectively overcome the problem of extremely low drug passing rate across the blood-brain barrier. Therefore, the peptide-lipid-(lipid-based carrier) conjugates of the present invention can be broadly used for the treatment and/or prevention of CNS diseases, and is also beneficial to the development of related clinical medical application.

Many changes and modifications in the above-described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A peptide-lipid conjugate, comprising:
a peptide consisting of an amino acid sequence selected from the group consisting of SEQ ID NO: 1 to SEQ ID NO: 29; and
a hydrophobic carbon chain conjugated to the peptide.

2. The peptide-lipid conjugate of claim 1, wherein a first carbon or a last carbon of the hydrophobic carbon chain is conjugated to a lysine of the peptide.

3. The peptide-lipid conjugate of claim 2, wherein the first carbon or the last carbon of the hydrophobic carbon chain is conjugated to a lysine of the peptide via a triazole moiety.

4. The peptide-lipid conjugate of claim 1, wherein the hydrophobic carbon chain comprises at least 6 carbons.

5. The peptide-lipid conjugate of claim 4, wherein the hydrophobic carbon chain is further conjugated to polyethylene glycol.

6. The peptide-lipid conjugate of claim 1, wherein the hydrophobic carbon chain is selected from dodecyne, cholesterol, and cholesterol-PEG.

7. The peptide-lipid conjugate of claim 1, further comprising a lipid-based carrier, wherein the hydrophobic carbon chain anchors into a lipid layer of the lipid-based carrier.

8. The peptide-lipid conjugate of claim 7, wherein the lipid-based carrier is selected form liposome, PEGylated liposome, cationic liposome, immunoliposome, transfersome, ethosome, niosome, virosome, nanoparticle, lipid nanoparticle, lipid nanoemulsion, microemulsion, pickering emulsion, micelle, cubosome, hexosome, spongosome, lipid-polymer hybrid nanoparticle, exosome, endosome, and extracellular vesicle.

9. The peptide-lipid conjugate of claim 7, wherein at least one reagent is encapsulated in the lipid-based carrier.

10. The peptide-lipid conjugate of claim 9, wherein the at least one reagent is selected from siRNA, shRNA, microRNA, double-stranded RNA, single-stranded RNA, DNA, aptamers, peptides, proteins, small chemical molecules, large chemical molecules, viral particles or fragments thereof, dendrimers, ligands, eukaryotic cells or fragments thereof, and prokaryotic cells or fragments thereof.

11. A method of transporting at least one reagent to a target, comprising binding a peptide-lipid-(lipid-based carrier) conjugate to a transferrin receptor, wherein the peptide-lipid-(lipid-based carrier) conjugate comprises:
a peptide consisting of an amino acid sequence selected from the group consisting of SEQ ID NO: 1 to SEQ ID NO: 29;
a hydrophobic carbon chain conjugated to the peptide; and
a lipid-based carrier encapsulating the at least one reagent;
wherein a first carbon or a last carbon of the hydrophobic carbon chain is conjugated to a lysine of the peptide, and the hydrophobic carbon chain anchors into a lipid layer of the lipid-based carrier.

12. The method of claim 11, wherein the hydrophobic carbon chain comprises at least 6 carbons.

13. The method of claim 12, wherein the hydrophobic carbon chain is further conjugated to polyethylene glycol.

14. The method of claim 11, wherein the lipid-based carrier is selected form liposome, PEGylated liposome, cationic liposome, immunoliposome, transfersome, ethosome, niosome, virosome, nanoparticle, lipid nanoparticle, lipid nanoemulsion, microemulsion, pickering emulsion, micelle, cubosome, hexosome, spongosome, lipid-polymer hybrid nanoparticle, exosome, endosome, and extracellular vesicle.

15. The method of claim 11, wherein the at least one reagent is selected from siRNA, shRNA, microRNA, double-stranded RNA, single-stranded RNA, DNA, aptamers, peptides, proteins, small chemical molecules, large chemical molecules, viral particles or fragments thereof, dendrimers, ligands, eukaryotic cells or fragments thereof, and prokaryotic cells or fragments thereof.

16. The method of claim 11, wherein the at least one reagent has to be transported across a tissue barrier to arrive at the target, the transferrin receptor is located on a cell of the tissue barrier, and binding of the peptide-lipid-(lipid-based carrier) conjugate to the transferrin receptor induces transcytosis of the cells, thereby transporting the peptide-lipid-(lipid-based carrier) conjugate across the tissue barrier to arrive at the target.

17. The method of claim 16, wherein the tissue barrier is one of a blood-brain barrier (BBB), a mucosal barrier, a gastrointestinal barrier, a blood retina barrier (BRB), a blood-thymus barrier (BTB), a blood-testis barrier (BTB), and a blood-placenta barrier (BPB).

18. The method of claim 11, wherein the target is a cell expressing the transferrin receptor, the transferrin receptor is located on a cell membrane of the cell, and binding of the peptide-lipid-(lipid-based carrier) conjugate to the transferrin receptor induces transcytosis of the cells, thereby transporting the peptide-lipid-(lipid-based carrier) conjugate across the cell membrane to arrive into the cell.

19. The method of claim 18, wherein the target is a cancer cell or a tissue cell.

20. The method of claim 11, wherein the target is in vivo or in vitro.


SEQUENCE LISTING (29 sequences)

SEQ ID NO: 1 - protein, 10 aa, synthetic construct
DIHVLDKEYD

SEQ ID NO: 2 - protein, 10 aa, synthetic construct
DISVLMKEYD

SEQ ID NO: 3 - protein, 10 aa, synthetic construct
DIDVLMKEYD

SEQ ID NO: 4 - protein, 10 aa, synthetic construct
DIHVLMKEYD

SEQ ID NO: 5 - protein, 10 aa, synthetic construct
DILVLMKEYD

SEQ ID NO: 6 - protein, 10 aa, synthetic construct
DIPVLMKEYD

SEQ ID NO: 7 - protein, 10 aa, synthetic construct
DITVLMKEYD

SEQ ID NO: 8 - protein, 10 aa, synthetic construct
DISLLMKEYD

SEQ ID NO: 9 - protein, 10 aa, synthetic construct
DISVIMKEYD

SEQ ID NO: 10 - protein, 10 aa, synthetic construct
DISVVMKEYD

SEQ ID NO: 11 - protein, 10 aa, synthetic construct
DISVLDKEYD

SEQ ID NO: 12 - protein, 10 aa, synthetic construct
DISVLNKEYD

SEQ ID NO: 13 - protein, 10 aa, synthetic construct
DISVLCKEYD

SEQ ID NO: 14 - protein, 10 aa, synthetic construct
DISVLKKEYD

SEQ ID NO: 15 - protein, 10 aa, synthetic construct
DISVLEKEYD

SEQ ID NO: 16 - protein, 10 aa, synthetic construct
DISVLQKEYD

SEQ ID NO: 17 - protein, 10 aa, synthetic construct
DISVLMKEYN

SEQ ID NO: 18 - protein, 10 aa, synthetic construct
DISVLMKEYE

SEQ ID NO: 19 - protein, 10 aa, synthetic construct
DISVLMKEYQ

SEQ ID NO: 20 - protein, 10 aa, synthetic construct
DISVLSKEYD

SEQ ID NO: 21 - protein, 10 aa, synthetic construct
DISVLMKDYD

SEQ ID NO: 22 - protein, 10 aa, synthetic construct
DISVLMKEFD

SEQ ID NO: 23 - protein, 10 aa, synthetic construct
DISVLMKESD

SEQ ID NO: 24 - protein, 10 aa, synthetic construct
DISVLMKETD

SEQ ID NO: 25 - protein, 10 aa, synthetic construct
NISVLMKEYD

SEQ ID NO: 26 - protein, 10 aa, synthetic construct
DISELMKEYD

SEQ ID NO: 27 - protein, 10 aa, synthetic construct
DISVLLKEYD

SEQ ID NO: 28 - protein, 10 aa, synthetic construct
DISVLMKHYD

SEQ ID NO: 29 - protein, 10 aa, synthetic construct
DITVLCKEYN